(19) United States
(12) Patent Application Publication
(10) Pub. No.: US 2004/0132823 A1
(43) Pub. Date: Jul. 8, 2004

(54) Pharmaceutical composition of 2-(4-isobutylphenyl) propionic acid

(76) Inventor: Leo Pavliv, Cary, NC (US)

Correspondence Address: Finnegan, Henderson, Farabow,, Garrett & Dunner, L.L.P., 1300 I Street, N.W., Washington, DC, 20005-3315, US

(21) Appl. No.: 10/739,050

(22) Filed: Dec. 19, 2003

Related U.S. Application Data
(63) Continuation-in-part of application No. 09/985,246, filed on Nov. 2, 2001, now Pat. No. 6,727,286.

(51) Int. Cl. A61K 31/198

(52) U.S. Cl. 514/565; 514/570

(57) ABSTRACT

The present invention provides a pharmaceutical composition comprising an aqueous solution of arginine and ibuprofen, wherein the molar ratio of arginine to ibuprofen is less than 1:1, as well as a method of making the same. The present invention also provides a pharmaceutical composition comprising an aqueous solution of arginine and ibuprofen, wherein the solution has a pH of less than about 7.8, as well as a method of making the same. The present invention also provides a method of treating a condition chosen from pain, inflammation, fever, and/or other conditions alleviated by ibuprofen comprising administering a pharmaceutical composition comprising an aqueous solution of arginine and ibuprofen, wherein the molar ratio of arginine to ibuprofen is less than 1:1, and wherein the molar ratio of arginine to ibuprofen is selected to achieve a pH of less than about 7.8.

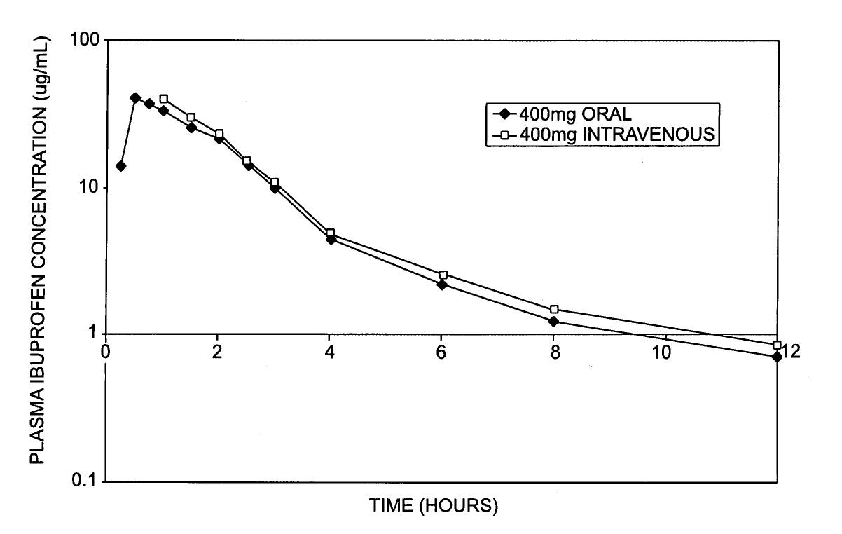

This application is a continuation-in-part application of application Ser. No. 09/985,246, which is hereby incorporated by reference in its entirety.

The present invention relates to a pharmaceutical composition for oral or injectable (parenteral) use containing 2-(4-isobutylphenyl)propionic acid and a basic amino acid, and more particularly, where the amino acid is arginine.

### BACKGROUND OF INVENTION

2-(4-isobutylphenyl)propionic acid, whose International Nonproprietary Name is ibuprofen, is a well-known anti-inflammatory drug having a molecular weight of 206.28 and the following chemical structure:

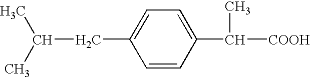

(Merck Index 12th ed., n4925, page 839). Originally patented in the 1960's, ibuprofen is now marketed generically, as well as under the tradenames of Motrin®, Advil®, and Nuprin® for the treatment of pain, inflammation, and fever.

Ibuprofen is readily available as the racemic mixture ((RS)-Ibuprofen) of the two enantiomers, (R)-Ibuprofen and (S)-Ibuprofen. Even though the (S) enantiomer is the biologically active form, most preparations contain the racemic mixture since the (R) enantiomer is converted to the active (S) form in-vivo. For simplicity, hereinafter the term “ibuprofen” will be used to indicate any one of the (R) enantiomer, the (S) enantiomer, or the racemate.

Many amino acids, including arginine, are available as both the D and L forms. For simplicity, hereinafter the term “arginine” will indicate the D or L form of arginine or a mixture of (D)-arginine and (L)-arginine. Arginine has a molecular weight of 174.20.

Although ibuprofen has many advantages over other analgesics such as aspirin and acetaminophen, it is very poorly soluble in water. Thus, certain dosage forms of ibuprofen, especially oral or injectable liquids, have been difficult to develop. Several U.S. patents have addressed this problem.

For example, U.S. Pat. No. 4,309,421 appears to describe water-soluble complexes of ibuprofen and phospholipids suitable for parenteral administration. U.S. Pat. Nos. 4,859,704 and 4,861,797 appear to describe the synthesis of alkali metal salts of ibuprofen for preparing a liquid ibuprofen formulation.

Other U.S. patents appear to address this problem by preparing an ibuprofen salt with a basic amino acid as the active pharmaceutical ingredient and then solubilizing the salt to produce a liquid dosage form.

For example, U.S. Pat. No. 5,200,558 appears to describe enhanced analgesic effects of S (+) ibuprofen as salts of L and D amino acids, including arginine, in various dosage forms, including as an injectable solution. U.S. Pat. No. 4,279,926 appears to describe the use of basic amino acid salts of propionic acids for relieving pain and treating inflammatory conditions. Similarly, U.S. Pat. No. 5,463,117 appears to describe the preparation of salts of ibuprofen with basic amino acids. Finally, U.S. Pat. No. 6,005,005 appears to describe a liquid composition for oral use containing ibuprofen and arginine.

However, the approaches described in the patents discussed above have, among others, the disadvantage of requiring the formation of a salt before solubilization, where the salt must be isolated and tested prior to producing the dosage form. Additionally, the ibuprofen formulations resulting from those processes have at least a 1:1 molar ratio of amino acid to ibuprofen. It is beneficial from both a cost and development point to not have to form a salt and isolate and test it prior to producing the dosage form. It is also beneficial in most cases to minimize the amount of non-active components, including salts, used in therapeutic products in order to minimize potential side effects. Furthermore, for injectable products it is beneficial to produce a liquid dosage form of ibuprofen having a pH similar to that of blood (pH 7.4). Finally, it is beneficial for an injectable and oral product to have similar pharmacokinetics to minimize the need for dosage adjustments.

### SUMMARY OF THE INVENTION

The present invention utilizes arginine to solubilize ibuprofen during the manufacture of the pharmaceutical product instead of using a salt form of ibuprofen. Thus, an embodiment of the present invention is a pharmaceutical composition comprising an aqueous solution of arginine and ibuprofen, wherein the molar ratio of arginine to ibuprofen is less than 1:1, and wherein the pH of the solution is less than about 7.8. Another embodiment of the present invention is a method of making a pharmaceutical composition comprising an aqueous solution of arginine and ibuprofen, wherein the molar ratio of arginine to ibuprofen is less than 1:1, and wherein the pH of the solution is less than about 7.8. Still other embodiments of the present invention are directed to methods of treating pain, inflammation, fever, and/or other conditions alleviated by ibuprofen comprising administering a pharmaceutical composition comprising an aqueous solution of arginine and ibuprofen, wherein the molar ratio of arginine to ibuprofen is less than 1:1, and wherein the pH of the solution is less than about 7.8.

### BRIEF DESCRIPTION OF THE FIGURE

The FIGURE shows plasma concentration-time curves for 400 mg oral and intravenous ibuprofen.

### DETAILED DESCRIPTION OF THE INVENTION

The present inventor has discovered that a liquid composition of ibuprofen can be produced by combining ibuprofen with arginine at molar ratios that minimize the amount of arginine necessary to solubilize the ibuprofen. Thus, one embodiment of the present invention is a pharmaceutical composition comprising an aqueous solution of arginine and ibuprofen, wherein the molar ratio of arginine to ibuprofen is less than 1:1. Another embodiment of the invention is a pharmaceutical composition comprising an aqueous solution of arginine and ibuprofen, wherein the molar ratio of arginine to ibuprofen is less than about 0.98:1. In another embodiment of the invention, the molar ratio of arginine to ibuprofen is from about 0.10:1 to about 0.999:1. In a further embodiment of the invention, the molar ratio of arginine to ibuprofen is from about 0.97:1 to about 0.98:1. In yet other embodiments of the invention, the molar ratio of arginine to ibuprofen is about 0.97:1 or about 0.92:1 or about 0.60:1 or about 0.99:1. In still yet other embodiments of the invention, the molar ratio of arginine to ibuprofen is about 0.90:1 or about 0.91:1 or about 0.93:1 or about 0.94:1 or about 0.95:1 or about 0.96:1 or about 0.98:1.

The present inventor has further discovered that a liquid composition of ibuprofen can be produced by combining ibuprofen with arginine at molar ratios that minimize the amount of arginine necessary to solubilize the ibuprofen, and that achieve a composition having a pH that is suitable for injection. Thus, another embodiment of the invention is a pharmaceutical composition comprising an aqueous solution of arginine and ibuprofen, wherein the pH is less than about 7.8. In yet another embodiment of the invention, the pharmaceutical composition comprises an aqueous solution of arginine and ibuprofen, wherein the pH is from about 7.6 to about 7.8. A further embodiment of the invention is a pharmaceutical composition comprising an aqueous solution of arginine and ibuprofen, wherein the pH is about 7.6. In still other embodiments, the pH of the solution is about 7.3 or about 7.4 or about 7.5 or about 7.7.

The present inventor has further discovered a method of making a pharmaceutical composition comprising an aqueous solution of arginine and ibuprofen in a molar ratio of less than 1:1, wherein the method comprises the following: adding arginine to water, mixing until the arginine is dissolved to form an arginine solution, adding ibuprofen to the arginine solution, and mixing until the ibuprofen is dissolved to form the aqueous solution of arginine and ibuprofen, optionally adding sufficient water to result in the desired concentration of ibuprofen, and optionally separating any precipitate using standard methods such as filtration or centrifugation. The resulting product is a clear, colorless solution that can readily be passed through a 0.2 micron filter. The pH of the resulting solution can be adjusted using techniques known in the art to achieve a desired pH, for example a pH similar to that of blood. Finally, the resulting solution can be terminally sterilized or lyophilized.

Alternatively, the ibuprofen can be added prior to the arginine, or the arginine and ibuprofen can be added at the same time. Moreover, the pH of the solution can be adjusted by adding additional arginine or ibuprofen to achieve the desired pH. For example, in one embodiment of the invention, an aqueous solution of arginine and ibuprofen is prepared that results in a molar ratio of less than 1:1, and then additional arginine is added to achieve a pH of about 7.6 to about 7.8.

The present inventor has further discovered a method of treating a condition chosen from pain, inflammation, fever, and/or other conditions alleviated by ibuprofen comprising administering to a patient in need thereof an effective amount of a pharmaceutical composition comprising an aqueous solution of arginine and ibuprofen, wherein the molar ratio of arginine to ibuprofen is less than 1:1. Additionally, the present inventor has discovered a method of treating a condition chosen from pain, inflammation, fever, and/or other conditions alleviated by ibuprofen comprising administering to a patient in need thereof an effective amount of a pharmaceutical composition comprising an aqueous solution of arginine and ibuprofen, wherein pH of the aqueous solution is less than about 7.8. Other conditions alleviated by ibuprofen include, but are not limited to, patent ductus arteriosis and certain forms of cancer. The pharmaceutical composition may be administered by injection (intravenous or intramuscular) or orally. Dosages of the pharmaceutical composition range from about 5 to about 1000 mg of ibuprofen in the pharmaceutical composition and can be determined by one of ordinary skill in the art. In one embodiment, the dosage is from about 100 to about 800 mg of ibuprofen in the pharmaceutical composition. In a further embodiment, the dosage is about 400 mg of ibuprofen in the pharmaceutical composition. In still another embodiment, the dosage of the pharmaceutical composition is from about 5 to about 10 mg/kg, and in a further embodiment the dosage of the pharmaceutical composition is about 7.5 mg/kg.

The following examples represent specific embodiments of the foregoing discovery, and they are not representative of the entire scope of the invention. The ibuprofen and arginine used in the examples are United States Pharmacopoea grade, but other pharmaceutically acceptable materials can be utilized.

### EXAMPLE 1

Add 8.2 kg of arginine to approximately 80 liters of water for injection and mix until dissolved. Add 10.0 kg of ibuprofen to the arginine solution and mix until dissolved. Add a sufficient quantity of water to equal 100 liters, resulting in a 100 mg/mL solution having a molar ratio of 0.97:1 (arginine:ibuprofen). The product results in a clear, colorless, solution that can readily be passed through a 0.2 micron filter. The pH of the resulting solution is approximately 7.4 and can be adjusted to achieve somewhat lower or higher pH's as desired. The solution can further be terminally sterilized to minimize the likelihood of a non-sterile product.

### EXAMPLE 2

Lower concentrations of ibuprofen can be prepared by using lesser amounts of arginine and ibuprofen. Add 41 g of arginine to approximately 80 liters of water for injection and mix until dissolved. Add 50 g of ibuprofen to the arginine solution and mix until dissolved. Add a sufficient quantity of water to equal 100 liters, resulting in a 0.5 mg/mL solution having a molar ratio of 0.97:1 (arginine:ibuprofen). The product results in a clear, colorless, solution that can readily be passed through a 0.2 micron filter. The pH of the resulting solution can be adjusted to achieve a desirable pH.

### EXAMPLE 3

Lower concentrations of arginine can be used to prepare the ibuprofen solution. Add 3.8 kg of arginine to approximately 80 liters of water for injection and mix until dissolved. Add 7.5 kg of ibuprofen to the arginine solution and mix until dissolved. Add a sufficient quantity of water to equal 100 liters, resulting in a 75 mg/mL solution having a molar ratio of 0.60:1 (arginine:ibuprofen). The product can be passed through a 0.2 micron filter resulting in a clear colorless solution. The pH of the resulting solution can be adjusted to achieve a desirable pH.

### EXAMPLE 4

Higher concentrations of arginine can be used to prepare the ibuprofen solution. Add 8.43 g of arginine to 80 mL of water for injection and mix until dissolved. Add 10 g of ibuprofen to the arginine solution and mix until dissolved. Add a sufficient quantity of water to equal 100 mL, resulting in a 100 mg/mL solution having a molar ratio of 0.99:1 (arginine:ibuprofen). The product results in a clear, colorless, solution that can readily be passed through a 0.2 micron filter. The pH of the resulting solution can be adjusted to achieve a desirable pH.

### EXAMPLE 5

4.384 kg of arginine were added to approximately 45 liters of water for injection and mixed until dissolved. 5.62 kg of ibuprofen were added to the arginine solution and mixed until dissolved. The pH of the resulting solution was approximately 7.4, but could be adjusted to achieve somewhat lower or higher pH's as desired. A sufficient quantity of water was added to the resulting solution to equal 56.2 liters, resulting in a 100 mg/mL solution having a molar ratio of 0.92:1 (arginine:ibuprofen). The product resulted in a clear, colorless solution that could readily be passed through a 0.2 micron filter. The solution was terminally sterilized to assure that the product was sterilized.

### EXAMPLE 6

In an attempt to demonstrate similar pharmacokinetics between a 60 minute infusion of intravenous ibuprofen solubilized with arginine as in Example 5 and oral ibuprofen (in the form of Advil® Liqui-Gels®), volunteers received single oral or intravenous doses (200 mg, 400 mg, or 800 mg) of either oral or intravenous ibuprofen product. Blood samples were collected at specified times relative to the start of dosing, and plasma ibuprofen concentrations were measured. The following pharmacokinetic parameters were calculated: Cₘₐₓ(maximum concentration), AUC₀₋₁₂ (area under the curve from initial time to 12 hours), AUC_{0-∞}, (area under the curve from initial time to infinity), Tₘₐₓ (time of maximum concentration), ke (elimination constant), and t_{1/2}(half life). Statistical analyses were performed on the plasma concentration data and pharmacokinetic parameters were calculated for the 12 patients on each of the three doses examined.

The plasma concentration-time profiles for both oral and intravenous administration of ibuprofen were observed to be very similar. The concentration-time data for the 400-mg oral and intravenous doses are shown in the FIGURE to illustrate this result. On the basis of the ibuprofen concentration-time data, the following pharmacokinetic parameters were calculated (Table 1).

TABLE 1
Pharmacokinetic Parameters After Oral and Intravenous Administration of Ibuprofen

| | Cohort 1: 200 mg | | Cohort 2: 400 mg | | Cohort 3: 800 mg | |
|---|---|---|---|---|---|---|
| Parameter | Oral | Intravenous | Oral | Intravenous | Oral | Intravenous |
| Cₘₐₓ | 24.7 ± 4.2 | 19.3 ± 3.1 | 42.9 ± 4.9 | 39.2 ± 6.1 | 81.0 ± 18.8 | 72.6 ± 9.6 |
| (μg/mL) | | | | | | |
| AUC₀₋₁₂ | 67.9 ± 16.9 | 63.2 ± 12.5 | 108.0 ± 23.9 | 108.5 ± 29.0 | 211.0 ± 47.6 | 192.2 ± 35.9 |
| (μg · hr/mL) | | | | | | |
| AUC_{0-∞} | 69.9 ± 18.0 | 65.5 ± 14.1 | 110.8 ± 26.8 | 112.3 ± 32.8 | 218.4 ± 55.0 | 197.8 ± 39.9 |
| (μg · hr/mL) | | | | | | |
| Tₘₐₓ (hr) | 0.6 ± 0.2 | 1.1 ± 0.2 | 0.6 ± 0.1 | 1.1 ± 0.2 | 0.9 ± 0.5 | 1.0 ± 0.0 |
| kₑₗ (hr⁻¹) | 0.3 ± 0.0 | 0.3 ± 0.0 | 0.3 ± 0.1 | 0.3 ± 0.1 | 0.3 ± 0.1 | 0.3 ± 0.0 |
| t_{1/2} (hr) | 2.4 ± 0.2 | 2.3 ± 0.2 | 2.2 ± 0.5 | 2.2 ± 0.5 | 2.3 ± 0.5 | 2.3 ± 0.4 |

Data shown are mean ± standard deviation.

The linearity of ibuprofen pharmacokinetics after oral and intravenous administration was analyzed. The results indicated that for both intravenous ibuprofen and oral ibuprofen, AUC₀₋₁₂, AUC_{0-∞}, and Cₘₐₓ increased in an appropriately linear manner with dose.

### EXAMPLE 7

Add 8.2 kg of arginine to approximately 60 liters of water for injection and mix until dissolved. Add 10.0 kg of ibuprofen to the arginine solution and mix until resulting solution is clear and ibuprofen has been dissolved. Add additional arginine to achieve a pH of about 7.6 to about 7.8. Add a sufficient quantity of water to equal 100 liters, resulting in a 100 mg/mL ibuprofen solution. The product results in a clear, colorless, solution that can readily be passed through a 0.2 micron filter. The solution can further be terminally sterilized to minimize the likelihood of a non-sterile product.

### EXAMPLE 8

Five aqueous solutions of arginine and ibuprofen were prepared, each with a different molar ratio of arginine to ibuprofen. The first column of Table 2 identifies the molar ratio of each aqueous solution. In order to prepare each of those solutions, the amount of arginine (in grams) identified in the second column of Table 2 was added to approximately 15 to 20 mL of water while stirring until the arginine was completely dissolved. Next, for each solution, 2.500 g of ibuprofen was added, as indicated in the third column of Table 2, and mixed until dissolved. Finally, a sufficient amount of water was added to each solution to attain a final volume of 25 mL for each solution. The concentrations of all solutions were 100 mg/mL ibuprofen. The five solutions were visually compared, and all resulted in clear colorless aqueous solutions with no visible precipitate. A standard pH meter calibrated with appropriate pH standards was then used to measure the pH of each solution. The results are shown for each solution in the final column of Table 2. Table 2. Compositions comprising arginine and ibuprofen and their associated pH Molar ratio Amount of Amount of pH (arginine: ibuprofen) Arginine (g) Ibuprofen (g)

TABLE 2
Compositions comprising arginine and
ibuprofen and their associated pH

| Molar ratio | Amount of | Amount of | |
|---|---|---|---|
| (arginine:ibuprofen) | Arginine (g) | Ibuprofen (g) | pH |
| 0.9:1 | 1.901 | 2.500 | 7.1 |
| 0.92:1 | 1.950 | 2.500 | 7.2 |
| 0.95:1 | 2.006 | 2.500 | 7.4 |
| 1:1 | 2.112 | 2.500 | 7.8 |
| 1.05:1 | 2.217 | 2.500 | 8.3 |

The results of the comparative test illustrated in Table 2 show that the presently claimed invention, i.e., compositions comprising an aqueous solution of arginine and ibuprofen, wherein the molar ratio of arginine to ibuprofen is less than 1:1, unexpectedly resulted in solutions exhibiting both: (1) complete dissolution of ibuprofen and (2) pH values of less than 7.8. This unexpected result is in contrast to the compositions with a molar ratio of equal to or greater than 1:1, which yielded pH values of greater than or equal to 7.8.

I claim:

1. A pharmaceutical composition comprising an aqueous solution of arginine and ibuprofen, wherein the pH of the aqueous solution of arginine and ibuprofen is less than about 7.8.

2. The pharmaceutical composition of claim 1, wherein the aqueous solution of arginine and ibuprofen has been terminally sterilized.

3. The pharmaceutical composition of claim 1, wherein the aqueous solution of arginine and ibuprofen has been lyophilized.

4. The pharmaceutical composition of claim 1, wherein the ibuprofen is (RS)-Ibuprofen.

5. The pharmaceutical composition of claim 1, wherein the ibuprofen is (S)-Ibuprofen.

6. The pharmaceutical composition of claim 1, wherein the arginine is L-arginine.

7. The pharmaceutical composition of claim 1, wherein the arginine is D-arginine.

8. The pharmaceutical composition of claim 1, wherein the molar ratio of arginine to ibuprofen is less than about 0.98:1.

9. The pharmaceutical composition of claim 8, wherein the molar ratio of arginine to ibuprofen is from about 0.97:1 to about 0.98:1.

10. The pharmaceutical composition of claim 9, wherein the molar ratio of arginine to ibuprofen is about 0.98:1.

11. The pharmaceutical composition of claim 8, wherein the molar ratio of arginine to ibuprofen is about 0.95:1.

12. The pharmaceutical composition of claim 1, wherein the pH of the aqueous solution of arginine and ibuprofen is from about 7.6 to about 7.8.

13. The pharmaceutical composition of claim 12, wherein the pH of the aqueous solution of arginine and ibuprofen is about 7.6.

14. The pharmaceutical composition of claim 12, wherein the pH of the aqueous solution of arginine and ibuprofen is about 7.7.

15. A method of making a pharmaceutical composition comprising an aqueous solution of arginine and ibuprofen, the method comprising dissolving arginine and ibuprofen in water to form the aqueous solution of arginine and ibuprofen, wherein the molar ratio of arginine to ibuprofen is less than 1:1.

16. The method of claim 15, wherein the molar ratio of arginine to ibuprofen is less than about 0.98:1.

17. The method of claim 16, wherein the molar ratio of arginine to ibuprofen is from about 0.97:1 to about 0.98:1.

18. The method of claim 17, wherein the molar ratio of arginine to ibuprofen is about 0.98:1.

19. The method of claim 16, wherein the molar ratio of arginine to ibuprofen is about 0.95:1.

20. The method of claim 15, further comprising adding additional arginine or ibuprofen so that the pH of the aqueous solution of arginine and ibuprofen is from about 7.6 to about 7.8.

21. The method of claims 20, wherein the pH of the aqueous solution of arginine and ibuprofen is about 7.6.

22. The method of claims 20, wherein the pH of the aqueous solution of arginine and ibuprofen is about 7.7.

23. The method of claim 15, further comprising terminally sterilizing the aqueous solution of arginine and ibuprofen.

24. The method of claim 15, further comprising lyophilizing the aqueous solution of arginine and ibuprofen.

25. The method of claim 15, wherein the ibuprofen is (RS)-Ibuprofen.

26. The method of claim 15, wherein the ibuprofen is (S)-Ibuprofen.

27. The method of claim 15, wherein the arginine is L-arginine.

28. The method of claim 15, wherein the arginine is D-arginine.

29. A pharmaceutical composition comprising an aqueous solution of arginine and ibuprofen prepared according to the method of claim 15.

30. A pharmaceutical composition comprising an aqueous solution of arginine and ibuprofen prepared according to the method of claim 20.

31. A method of treating a condition chosen from pain, inflammation, fever, and patent ductus arteriosis, comprising administering to a patient in need thereof an effective amount of an aqueous solution of arginine and ibuprofen, wherein the pH of the aqueous solution of arginine and ibuprofen is less than about 7.8.

32. The method of claim 31, wherein the molar ratio of arginine to ibuprofen is less than about 0.98:1.

33. The method of claim 32, wherein the molar ratio of arginine to ibuprofen is from about 0.97:1 to about 0.98:1.

34. The method of claim 33, wherein the molar ratio of arginine to ibuprofen is from about 0.98:1.

33. The method of claim 32, wherein the molar ratio of arginine to ibuprofen about 0.95:1.

36. The method of claim 31, wherein the pH of the aqueous solution of arginine and ibuprofen is from about 7.6 to about 7.8.

37. The method of claim 36, wherein the pH of the aqueous solution of arginine and ibuprofen is about 7.6.

38. The method of claim 36, wherein the pH of the aqueous solution of arginine and ibuprofen is about 7.7.

39. The method of claim 31, wherein the administration occurs via intravenous injection.

40. The method of claim 31, wherein the administration occurs via intramuscular injection.

41. The method of claim 31, wherein the administration occurs orally.

42. The method of claim 31, wherein the effective amount is from about 100 mg to about 800 mg of ibuprofen.

43. The method of claim 42, wherein the effective amount is about 400 mg of ibuprofen.

44. The method of claim 31, wherein the effective amount is about 7.5 mg/kg of ibuprofen.

45. The method of claim 31, wherein the condition is pain.

46. The method of claim 31, wherein the condition is inflammation.

47. The method of claim 31, wherein the condition is fever.

48. The method of claim 31, wherein the condition is patent ductus arteriosis.
